(19) United States
(12) Patent Application Publication
(10) Pub. No.: US 2007/0258912 A1
(43) Pub. Date: Nov. 8, 2007

(54) Use of Fluorescent Whitening Agents as Antimicrobials

(76) Inventors: Werner Holzl, Eschentzwiller (FR); Andrea Preuss, Basel (CH); Olof Wallquist, Therwil (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 11/659,549

(22) PCT Filed: Aug. 3, 2005
(86) PCT No.: PCT/EP05/53780

§ 371 Date: Feb. 6, 2007

(30) Foreign Application Priority Data
Aug. 9, 2004 (EP) 0410380.8

(51) Int. Cl. A61K 8/00; A61K 31/10; A61K 31/135; A61K 31/41; A61Q 11/00; A61K 31/465; A61K 31/53

(52) U.S. Cl. 424/49; 514/245; 514/296; 514/312; 514/315; 514/332; 514/359; 514/375; 514/387; 514/406; 514/443; 514/457; 514/648; 514/709

(57) ABSTRACT

Use of fluorescent whitening agents for whitening and the antimicrobial treatment of surfaces, and preservation of cosmetics, household products, personal care products, textiles, paper and starting materials of paper, plastics and disinfectants.

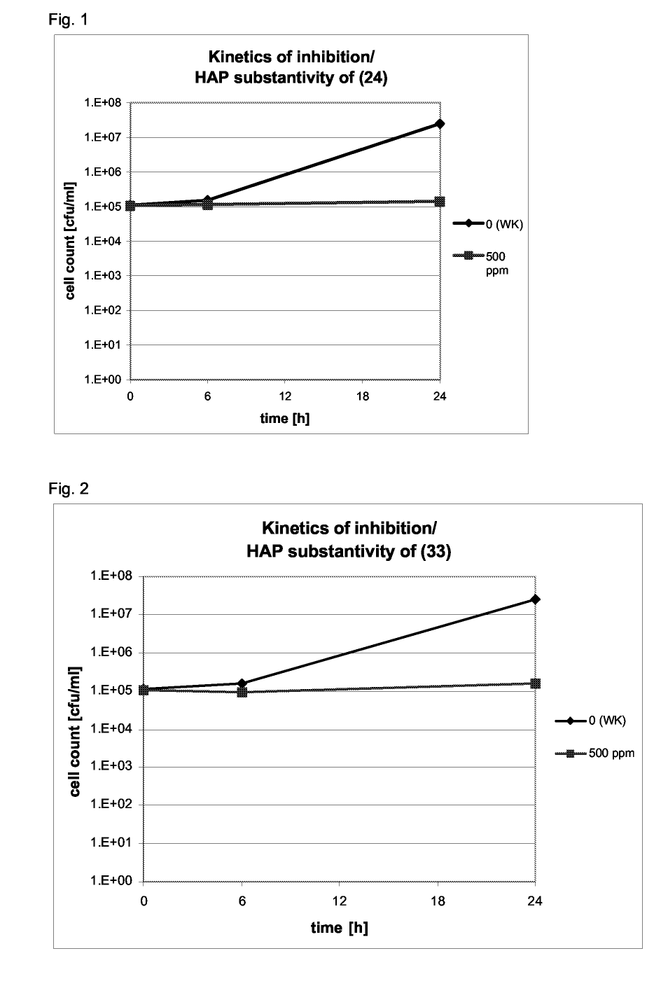

The present invention relates to the use of fluorescent whitening agents for whitening and the antimicrobial treatment of surfaces, and preservation of cosmetics, household products, personal care products, textiles, paper and starting materials of paper, plastics and disinfectants.

The present invention concerns the use of fluorescent whitening agent for the whitening of teeth and/or antimicrobial treatment of surfaces, especially teeth.

Preferred is the use of fluorescent whitening agent for the whitening of teeth with the proviso that a fluorescent whitening agent is not covered by a crosslinked polyvinyl alcohol shell.

Further, preferred fluorescent whitening agents are bis-triazinyl-diaminostilbene, 2-(stilbene-4-yl)-naphthatriazole, 2-(4-phenylstilbene-4-yl)benzoxazole, bis(azol-2-yl)stilbene, 1,4-bis(styryl)benzene, 4,4′-bis(styryl)biphenyl, 1,3-diphenyl-2-pyrazoline, bis(benzoxazol-2-yl), bis(benzimidazol-2-yl), 2-(benzofuran-2-yl)-benzimidazole, coumarine, carbostyrile, naphthalimide, quaternized pyridotriazole, pyrene derivatives or acylamino 3,7-diamino-dibenzothiophene-2,8-disulfonic acid 5,5-dioxide.

More preferred fluorescent whitening agents are those of formulae (1) to (20): bis-triazinyl-diaminostilbene of formula (1) 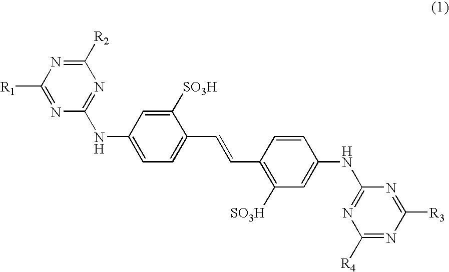
wherein
R₁, R₂, R₃ and R₄ are independently from each other NR₅R₆, OR₇ or a heterocyclic ring wherein
R₅ and R₆ are independently from each other hydrogen; substituted or unsubstituted C₆-C₁₀aryl, C₁-C₁₀alkyl, N,N′-diC₁-C₆alkylaminoC₁-C₁₀alkyl or a heterocyclic ring, and
R₇ is substituted or unsubstituted C₆-C₁₀aryl, C₁-C₁₀alkyl,
preferably
R₁, R₂, R₃ and R₄ are independently from each other substituted or unsubstituted phenyl, NH(C₁-C₆)alkyl, N,N′-diC₁-C₂alkylaminoC₁-C₆alkyl, NH(C₁-C₆)alkanol, NHphenyl, O(C₁-C₆)alkyl, NH₂, piperidine or morpholino and
R₇ is substituted or unsubstituted phenyl, C₁-C₆alkyl;
more preferably
R₁, R₂, R₃ and R₄ are independently from each other unsubstituted aryl; or with SO₃H/Na/K, COOR₇, CONHR₇, CON(R₇) substituted aryl; substituted or unsubstituted NH(C₁-C₆)alkyl, N((C₁-C₆)alkyl)₂, N,N′-diC₁-C₂alkylaminoC₁-C₃alkyl, NH(C₁-C₆)alkanol, N((C₁-C₆)alkanol)₂, NHphenyl, O(C₁-C₆)alkyl, NH₂, piperidine or morpholino and
R₇ is substituted or unsubstituted phenyl, C₁-C₄alkyl;
most preferably
R₁, R₂, R₃ and R₄ are independently from each other unsubstituted aryl; or with SO₃H/Na/K, COOR₇, CONHR₇, CON(R₇) substituted aryl, substituted or unsubstituted NH(C₁-C₂)alkyl, N((C₁-C₂)alkyl)₂, N,N′-dimethylaminopropyl, NHethanol, N(ethanol)₂, NHphenyl, O(C₁-C₆)alkyl, NH₂, or morpholino and
R₇ is substituted or unsubstituted phenyl, C₁-C₄alkyl;
and SO₃H can be the free sulfonic acid or an alkalimetal or earthalkalimetal salt
or
2-(stilbene-4-yl)-naphthatriazole of formula (2) 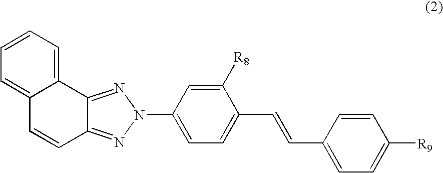
wherein
R₈ and R₉ are independently from each other hydrogen, SO₃H, CN, halogen;
preferably
R₈ and R₉ are independently from each other hydrogen, SO₃H, CN, or chloride; or
2-(4-phenylstilbene-4-yl)benzoxazole of formula (3) 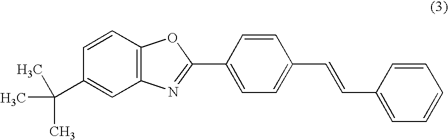
or
bis(azol-2-yl)stilbene of formula (4) 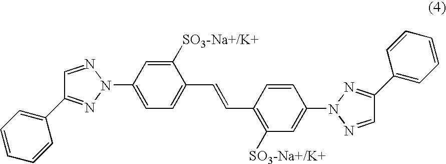
or
1, 4 or 2,3′ or 2,4′-bis(styryl)benzene of formula (5) 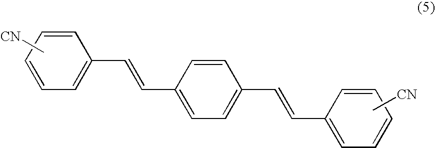
or
4,4-bis(styryl)biphenyl of formula (6) 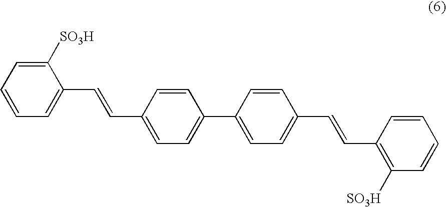
or
1,3-diphenyl-2-pyrazoline of formula (7) 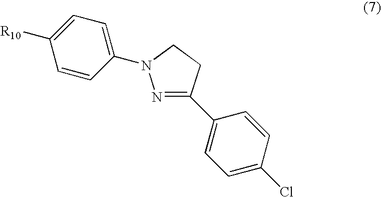
R₁₀ is SO₃H, SO₂NR₁₁R₁₂,
wherein
R₁₁ and R₁₂ are each independently from each other hydrogen, (C₁-C₆)alkyl-N⁺(C₁-C₆)alkyl, (C₁-C₆)alkyl-SO₃H;
preferably
R₁₀ is SO₃H, SO₂NH₂, SO₂NHCH₂CH₂CH₂N⁺(CH₃)₃, SO₂NHCH₂CH₂CH₂SO₃H,
or
bis-benzoxazole of formula (8) 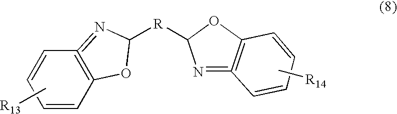
wherein
R₁₃ and R₁₄ are independently from each other hydrogen; substituted or unsubstituted C₆-C₁₀aryl, C₁-C₁₀alkyl, 1,2-diphenylvinyl, COO—C₁-C₁₀alkyl or SO₂—C₁-C₁₀alkyl, and
R is —C═C—, 1,2-dipenylvinylen, 1,4-naphthalen or 2,5-thiophenylene,
preferably
R₁₃ and R₁₄ are independently from each other hydrogen; substituted or unsubstituted phenyl, naphthyl, thiophenyl, C₁-C₁₆alkyl, 1,2-diphenylvinyl, COO—C₁-C₆alkyl or SO₂—C₁-C₆alkyl;
or most preferable 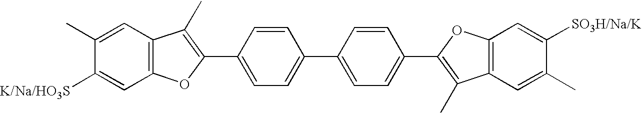
or
bis(benzimidazol-2-yl) of formula (9) 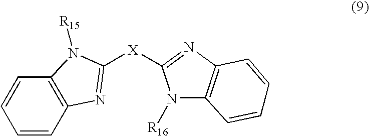
wherein
R₁₅ and R₁₆ are independently from each other hydrogen substituted or unsubstituted C₁-C₁₆alkyl or phenyl; and 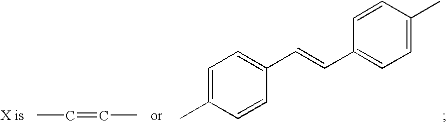
or
2-(benzofuran-2-yl)-benzimidazole of formula (10) 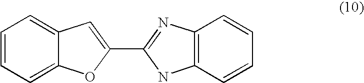
or
coumarines, including 3-phenyl-7-aminocoumarin, 3-phenyl-7-(azol-2-yl)coumarines, 3,7-bis(azolyl)-coumarines, and compounds of formulae (11), (12), (13) or (14) 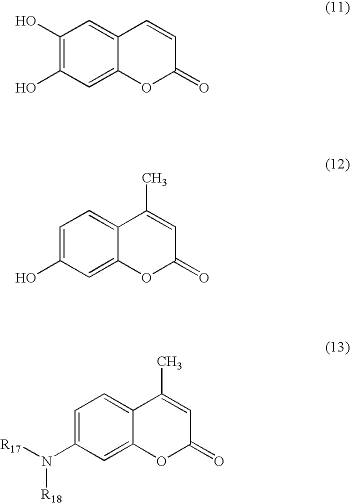
wherein
R₁₇ and R₁₈ are independently from each other hydrogen or substituted or unsubstituted C₁-C₆alkyl 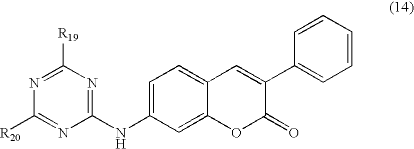
wherein
R₁₉ and R₂₀ are independently from each other NR₂₁R₂₂, OR₂₃ or a heterocyclic ring
wherein
R₂₁ and R₂₂ are independently from each other hydrogen; substituted or unsubstituted C₆-C₁₀aryl, C₁-C₁₀alkyl and
R₂₃ is substituted or unsubstituted C₆-C₁₀aryl, C₁-C₁₀alkyl;
or
carbostyrile of formula (15) 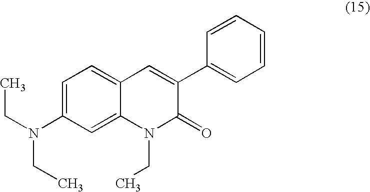
or
naphthalimide of formula (16) 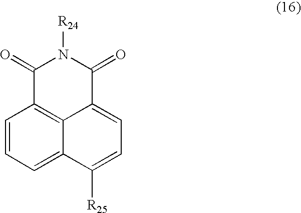
wherein
R₂₄ is hydrogen or substituted or unsubstituted C₁-C₁₆alkyl or phenyl, and
wherein
R₂₅ is hydrogen or substituted or unsubstituted NR₂₆R₂₇, OR₂₈ or a heterocyclic ring
wherein
R₂₆, R₂₇ and R₂₈ have the same definition as R₂₁, R₂₂ and R₂₃ as given above; or quaternized pyridotriazole;
or
pyrene of formula (17) 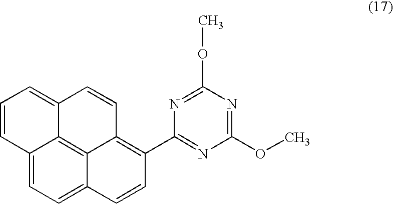
or
acylamino 3,7-diamino-dibenzothiophene-2,8-disulfonic acid 5,5-dioxide of formula (18) 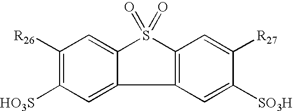
wherein
R₂₆ and R₂₇ are independently from each other substituted or unsubstituted CO-alkoxybenzoyl, CO—(C₁-C₆)alkyl, CO-phenyl,
or
bisstyrylbenzol of formula (19) 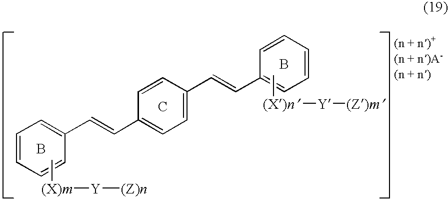
or
bisstyrylbiphenyl of formula (20) 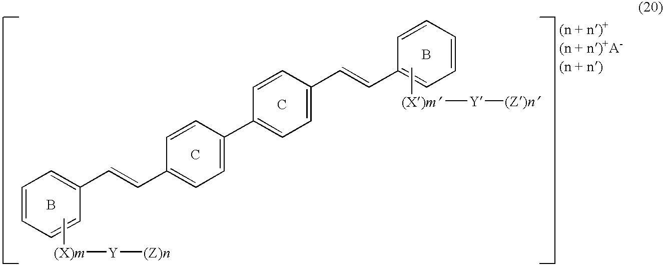
wherein
X and X′ independently of one another are —COO— or —CON(R₃₁), a direct bond, oxygen, sulfur, —O—C₁-C₃alkylene-CON(R₃₁)—, —SO₂N(R₃₁)—, —O—C₁-C₃-alkylene-COO— or —OCO—,
Y and Y′ independently of one another are a direct bond, C₁-C₂₀alkylene, a direct bond,
Z is pyridine, 2-pyridine-N-methyl, 4-pyridine-N-methyl or N(R₂₈R₂₉(R₃₀)_{q}), and
Z′ is pyridine, 2-pyridine-N-methyl, 4-pyridine-N-methyl or N(R_{28′}R_{29′}(R₃₀)_{q′}),
wherein
R₂₈ and R_{28′} independently of one another are unsubstituted or substituted C₁-C₈-alkyl or C₃-C₄alkenyl, or R₂₈ together with R₂₉, or R_{28′} together with R_{29′}, is a heterocyclic ring, R₂₉ and R_{29′} independently of one another are unsubstituted or substituted C₁-C₈alkyl or C₃-C₄alkenyl, or R₂₉ together with R₂₈ or R_{29′} together with R_{28′}, is a heterocyclic ring, or R₂₈ and R₂₉, or R_{28′} and R_{29′}, together with R₃₀ are a pyridine or picoline ring, R₃₀ is hydrogen, unsubstituted or substituted C₁-C₄alkyl or C₃-C₄alkenyl, or together with R₂₈ and R₂₉ or with R_{28′} and R_{29′} is a pyridine or picoline ring, R₃₀ is hydrogen or unsubstituted or substituted C₁-C₆-alkyl,
A⁻ is a colourless anion, and
n and n′ independently of one another are the number 0 or 1, and
m and m′ independently of one another are the number 0 or 1, and
p and p′ independently of one another are the number 0, 1, 2 or 3, and
q and q′ independently of one another are the number 0 or 1, and
the benzene nuclei B and C can also be substituted by non-chromophoric substituents;
preferably,
bisstyrylbenzol of formula (21) 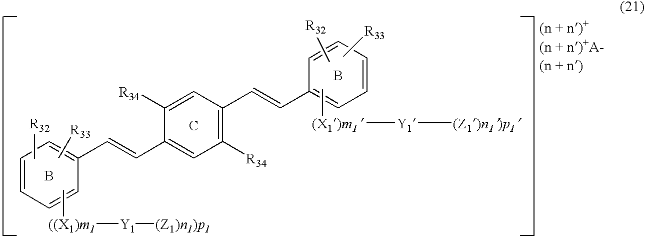
or
bisstyrylbiphenyl of formula (22) 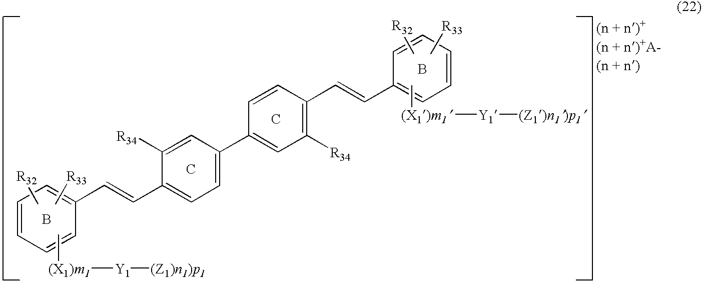
wherein
X₁ and X₁′ are —COO— or —CONH—, a direct bond, oxygen, sulfur, —OC₁-C₃alkylene-CONH—, —SO₂NH—, —O—C₁-C₃alkylene-COO— or —OCO—,
Y₁ and Y₁′ independently of one another are a direct bond, C₁-C₄alkylene or hydroxypropylene,
Z₁ and Z₁′ independently of one another are pyridine, 2-pyridine-N-methyl, 4-pyridine-N-methyl or N(R₃₅R₃₆(R₃₇)_{q″}),
wherein
R₃₅ and R₃₆ independently of one another are C₁-C₄alkyl or together are a pyrrolidine, piperidine, hexamethyleneimine or morpholine ring, or together with R₃₇ are a pyridine or picoline ring,
R₃₇ is hydrogen, C₁-C₄alkyl, C₃-C₄alkenyl, C₁-C₃alkoxycarbonylmethyl, benzyl, C₂-C₄-hydroxyalkyl or C₂-C₄cyanoalkyl, or together with R₃₅ and R₃₆ is a pyridine or picoline ring,
R₃₂ is hydrogen, chlorine, C₁-C₄alkyl, C₃-C₄alkenyl, C₁-C₃alkoxy, or (X₁)ₘ—Y₁—N(R₃₅R₃₆(R₃₇)_{q}—), or together with R₃₃ is a trimethylene or tetramethylene group,
R₃₃ is hydrogen, chlorine, C₁-C₄alkyl or C₁-C₃alkoxy, or together with R₃₂ is a trimethylene or tetramethylene group,
R₃₄ is hydrogen, chlorine or methyl,
n₁ and n_{1′} independently of one another are the number 0 or 1,
m₁ and m₁′ independently of one another are the number 0 or 1, and
p₁ and p₁′ independently of one another are the number 0, 1, 2 or 3, and
q″ is the number 0 or 1, and
A- is a colourless anion;
and
more preferred are bisstyrylbenzol of formula (40) 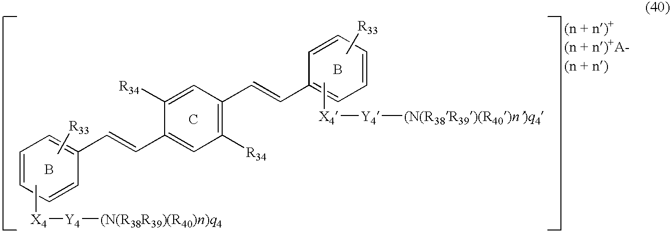
or
bisstyrylbiphenyl of formula (41) 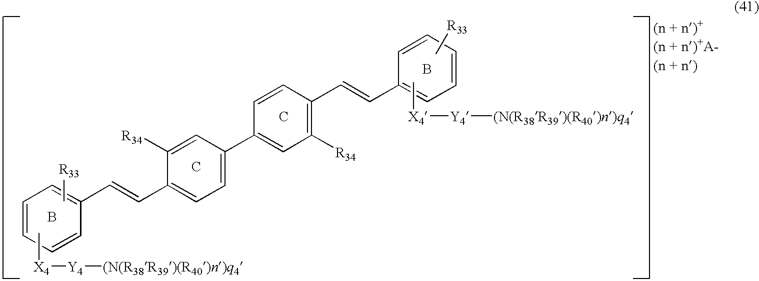
bisstyrylbenzol of formula (42) 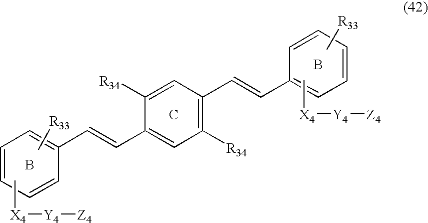
or
bisstyrylbiphenyl of formula (43) 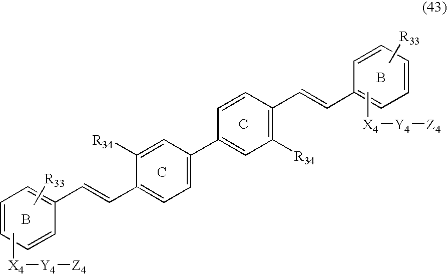
wherein
X₄ is oxygen, sulfur,
Y₄ is a direct bond C₁-C₄alkylene,
Z₄ is N(R₄₀R₄₁(R₄₂)_{q4}),
wherein
R₄₀ and R₄₁ independently of one another are C₁-C₄alkyl or together are a pyrrolidine, piperidine, hexamethyleneimine or morpholine ring, or together with R₄₂ are a pyridine or picoline ring,
R₄₂ is hydrogen or C₁-C₄alkyl,
R₃₃ is hydrogen, chlorine, C₁-C₄alkyl or C₁-C₃alkoxy,
R₃₄ is hydrogen, chlorine or methyl,
q4 is the number 0 or 1.

Further,

more preferred fluorescent whitening agents are those of formulae (1) to (4) and (7) to (10), (15), (17) to (20), within the above given definitions and preferences.

Most preferred fluorescent whitening agents are compounds of formulae (23) to (34)

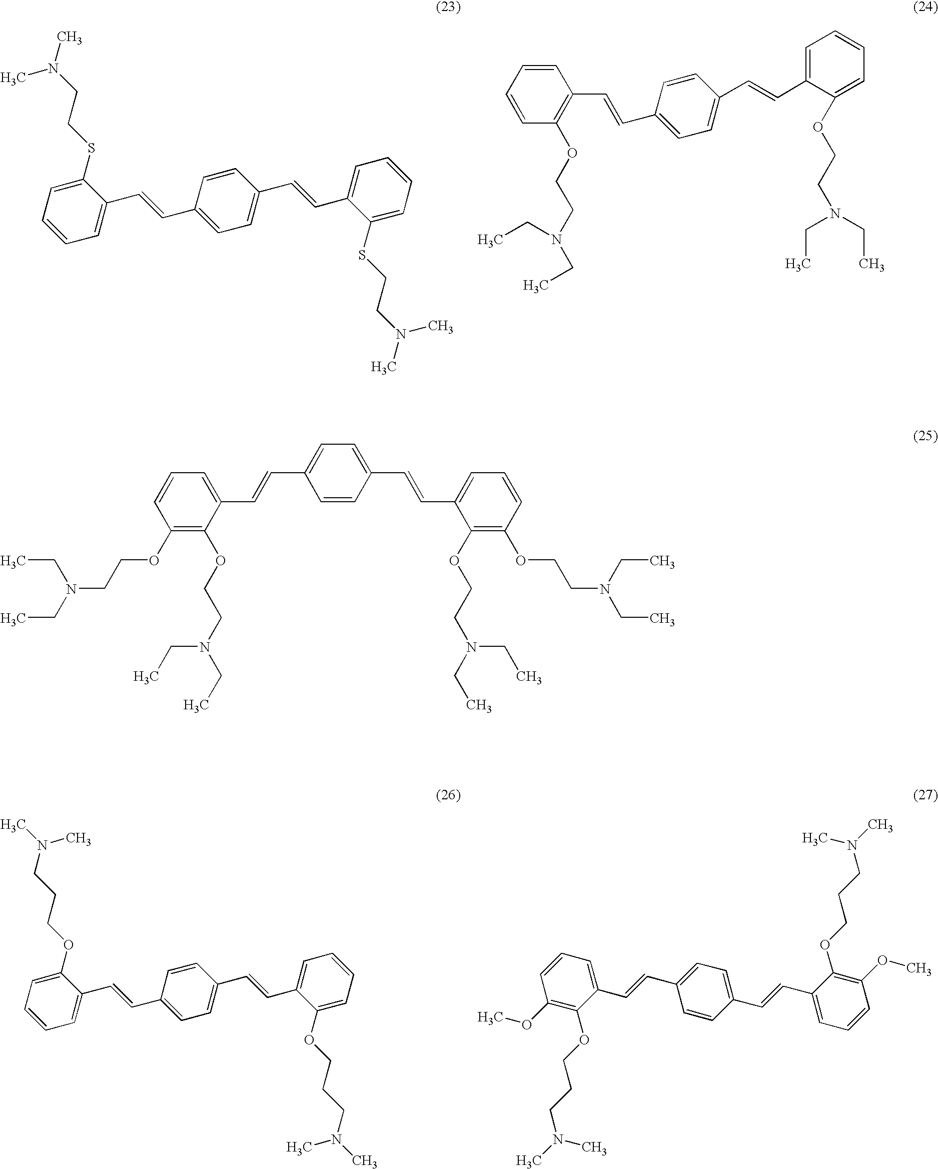

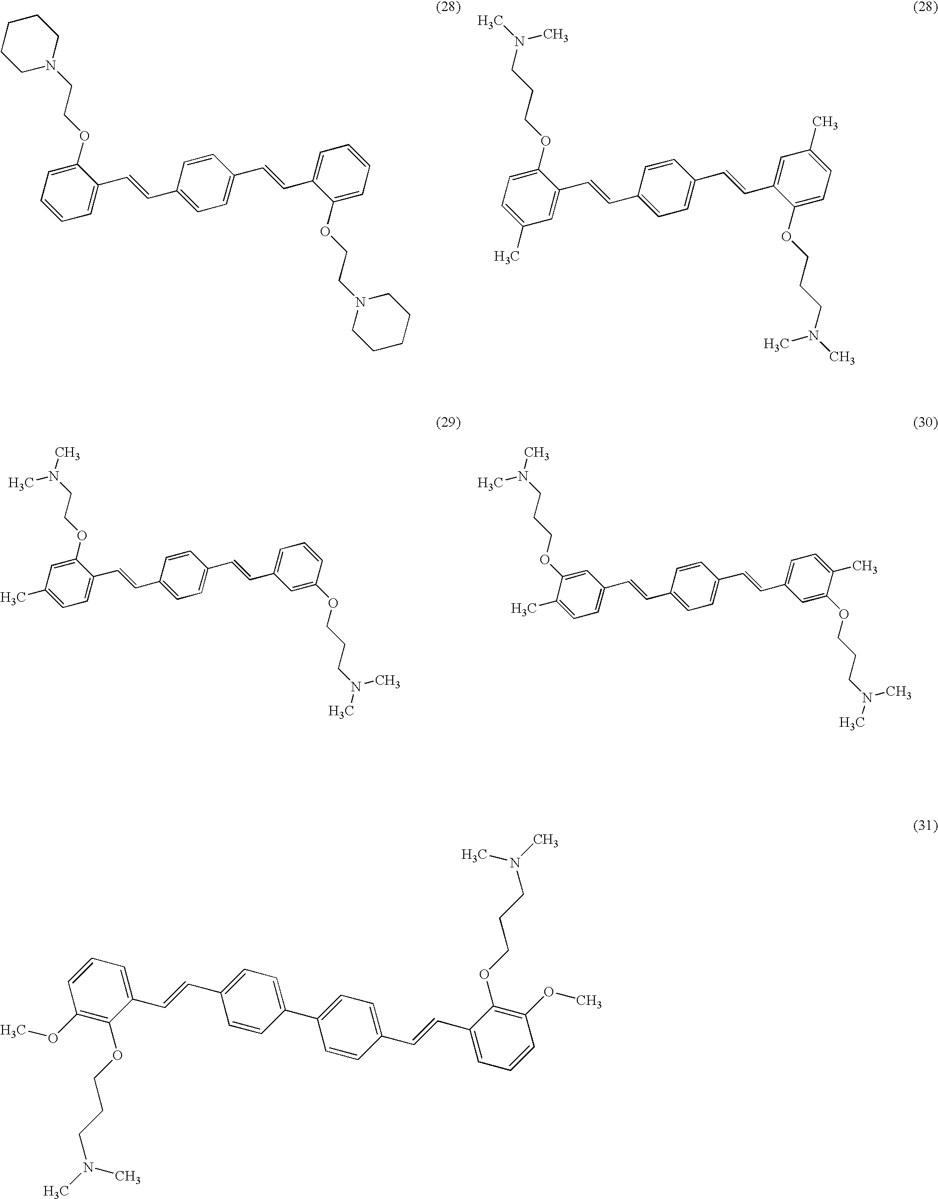

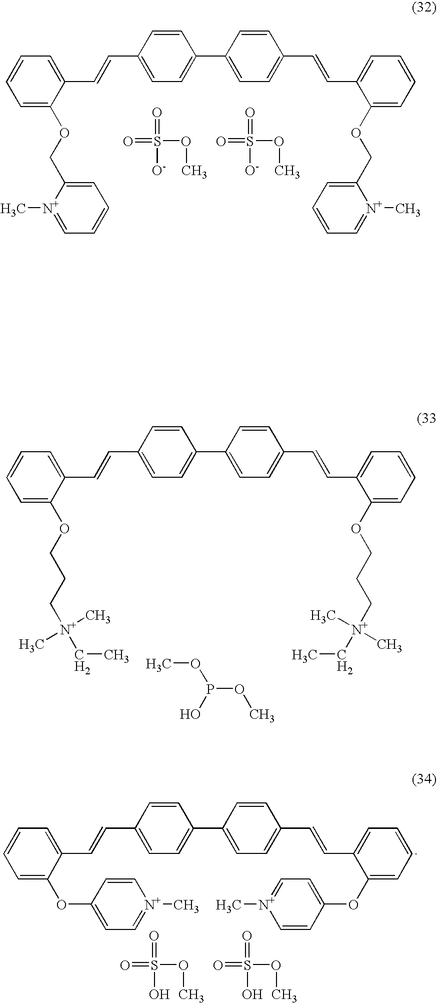

Especially preferred fluorescent whitening agents are compounds of formulae (26) and (28).

In the present invention one or more of the same or different substitutent chosen from the following group of substituents are for example suitable C₁-C₁₆alkyl, C₁-C₂₀alkylene, arylene or aryl-C₁-C₁₀alkylene, hydroxyl, C₁-C₈alkoxy, cyanide, halide, aryl, aralkyl, alkylaryl and NR40R41, wherein

R₄₀ and R₄₁ are each independently of the other hydrogen, unsubstituted or substituted aryl radical or C₁-C₆alkyl; or C₁-C₈alkyl, C₁-C₈alkoxy, cyanide and/or halide.

Preferred are R₄₀ and R₄₂ hydrogen or unsubstituted C₁-C₆alkyl.

Heterocyclic ring are an unsubstituted or substituted aromatic or non aromatic ring, such as for example thiophenyl, 1,3-thiazolyl, 1,2-thiazolyl, 1,3-benzothiazolyl, 2,3-benzothiazolyl, imidazolyl, 1,3,4-thiadiazolyl, 1,3,5-thiadiazolyl, 1,3,4-triazolyl, picoline, pyrazolyl, benzimidazolyl, benzopyrazolyl, morpholino, pyrrolidine, piperidine, hexamethyleneimine, 2-pyridine-N-methyl, 4-pyridine-N-methyl, pyridinyl, quinolinyl, pyrimidinyl and isoxazolyl, aminodiphenyl, aminodiphenylether or azobenzenyl.

Aryl is, for example, unsubstituted or substituted phenyl or naphthyl,

The substituted or unsubstituted alkylene or alkyl residues may be straight-chain, branched, or, from C₅alkyl upwards, monocyclic or polycyclic, and may be uninterrupted or interrupted by hetero atoms, such as such as O, S, CO, N, NH, NR₄₀; for example C₁-C₁₀alkylene may be a residue such as:

—CH₂CH₂—O—CH₂CH₂—O—CH₂CH₂—, or —CH₂CH₂—O—CH₂CH₂—, or —CH₂CH₂—O—CH₂—, or

—CH₂—O—CH₂—, or —CH₂CH₂—CH₂CH₂—O—CH₂—CH₂—, or —CH₂CH₂—CH(N(CH₃)₂)—CH₂—CH₂—, or

CH₂—NH₂—CH₂—CH₂.

C₁-C₁₆alkyl is, for example, methyl, ethyl, propyl, isopropyl, n-butyl, sec-butyl, tert-butyl, n-pentyl, 2-pentyl, 3-pentyl, 2,2′-dimethylpropyl, cyclopentyl, cyclohexyl, n-hexyl, n-octyl, 1,1′,3,3′-tetramethylbutyl or 2-ethylhexyl, nonyl, decyl, undecyl, dodecyl, tredecyl, tetradecyl, pentadecyl, hexadecyl.

C₁-C₁₀alkyl is, for example, methyl, ethyl, propyl, isopropyl, n-butyl, sec-butyl, tert-butyl, n-pentyl, 2-pentyl, 3-pentyl, 2,2′-dimethylpropyl, cyclopentyl, cyclohexyl, n-hexyl, n-octyl, 1,1′,3,3′-tetramethylbutyl or 2-ethylhexyl, nonyl, decyl.

C₁-C₈alkyl is, for example, methyl, ethyl, propyl, isopropyl, n-butyl, sec-butyl, tert-butyl, n-pentyl, 2-pentyl, 3-pentyl, 2,2′-dimethylpropyl, cyclopentyl, cyclohexyl, n-hexyl, n-octyl, 1,1′,3,3′-tetramethylbutyl or 2-ethylhexyl.

C₁-C₆alkyl is, for example, methyl, ethyl, propyl, isopropyl, n-butyl, sec-butyl, tert-butyl, n-pentyl, 2-pentyl, 3-pentyl, 2,2′-dimethylpropyl, cyclopentyl, cyclohexyl, n-hexyl.

C₁-C₄alkyl is, for example, methyl, ethyl, propyl, isopropyl, n-butyl, sec-butyl, tert-butyl.

NH(C₁-C₆)alkyl is, for example, NH-methyl, NH-ethyl, NH-propyl, NH-isopropyl, NH-n-butyl, NH-sec-butyl, NH-tert-butyl, NH-n-pentyl, NH-2-pentyl, NH-3-pentyl, NH-2,2′-dimethylpropyl, NH-cyclopentyl, NH-cyclohexyl, NH-n-hexyl.

NH(C₁-C₆)alkanol is, for example, NH-methanol, NH-ethanol, NH-propanol, NH-isopropanol, NH-n-butanol, NH-sec-butanol, NH-tert-butanol, NH-n-pentanol, NH-2-pentanol, NH-3-pentanol, NH-2,2′-dimethylpropanoyl, NH-cyclopentanol, NH-cyclohexanol, NH-n-hexanol.

N,N′-diC₁-C₆alkylaminoC₁-C₁₀alkyl is for example N,N′-dimethylaminomethyl, N,N′-diethylaminomethyl, N,N′-dimethylaminoethyl, N,N′-dipropylaminomethyl, N,N′-dipropylaminomethyl, N,N′-dipropylaminopropyl, N,N′-diisopropylaminomethyl, N,N′-diisopropylaminoetyl, N,N′-diisopropylaminopropyl or N,N′-dihexylaminomethyl, N,N′-dioctylaminoetyl, N,N′-dinonylaminopropyl.

N,N′-diC₁-C₂alkylaminoC₁-C₆alkyl is for example N,N′-dimethylaminomethyl, N,N′-diethylaminomethyl, N,N′-dimethylaminoethyl, N,N′-dimethylaminomethyl, N,N′-dimethylaminoisopropyl, N,N′-dimethylaminobutyl, N,N′-dimethylaminopentyl, N,N′-dimethylaminohexyl.

O-alkyl is the same as alkoxy and stands preferably for O(C₁-C₈)alkyl, O(C₁-C₆)alkyl, O(C₁-C₃)alkyl.

O(C₁-C₈)alkyl is, for example, methoxy, ethoxy, propoxy, isopropoxy, n-butoxy, sec-butoxy, tert-butoxy, n-pentoxy, 2-pentoxy, 3-pentoxy, 2,2′-dimethylpropoxy, cyclopentoxy, cyclohexoxy, n-hexoxy, n-heptoxy or n-octoxy.

O(C₁-C₆)alkyl is, for example, methoxy, ethoxy, propoxy, isopropoxy, n-butoxy, sec-butoxy, tert-butoxy, n-pentoxy, 2-pentoxy, 3-pentoxy, 2,2′-dimethylpropoxy, cyclopentoxy, cyclohexoxy, n-hexoxy.

O(C₁-C₃)alkyl is, for example, methoxy, ethoxy, propoxy, isopropoxy.

C₁-C₆alkanol is, for example, methanol, ethanol, propanol, isopropanol, n-butanol, sec-butanol, tert-butanol, n-pentanol, 2-pentanol, 3-pentanol, 2,2′-dimethylpropanol, cyclopentanol, cyclohexanol, n-hexanol.

C₁-C₂₀alkylene is, for example, methylene, ethylene, propylene, isopropylene, n-butylene, sec-butylene, tert-butylene, n-pentylene, 2-pentylene, 3-pentylene, 2,2′-dimethylpropylene, cyclopentylene, cyclohexylene, n-hexylene, n-octylene, 1,1′,3,3′-tetramethylbutylene or 2-ethylhexylene, nonylene, decylene, undecylene, dodecylene, tredecylene, tetradecylene, pentadecylene, hexadecylene, heptadecylene, ocatdecylene, nonadecylene.

C₁-C₄alkylene is, for example, methylene, ethylene, propylene, isopropylene, n-butylene, sec-butylene, tert-butylene.

C₁-C₃alkylene is, for example, methylene, ethylene, propylene, isopropylene.

C₃-C₄alkenyl is, for example, propenyl, isopropenyl, n-butenyl, sec-butenyl, tert-butenyl.

Halogen is, for example, fluoride, chloride, bromide or iodide, especially chloride and fluoride.

A⁻ is a colourless anion such as for example an organic or inorganic anion, such as halide, preferably chloride and fluoride, sulfate, hydrogen sulfate, phosphate, boron tetrafluoride, carbonate, bicarbonate, oxalate or C₁-C₈alkyl sulfate, especially methyl sulfate or ethyl sulfate; anion also denotes lactate, formate, acetate, propionate or a complex anion, such as the zinc chloride double salt.

The anion is especially a halide, preferably chloride or fluoride, sulfate, hydrogen sulfate, methylsulfate, phosphate, formate, acetate or lactate.

The anion is more especially fluoride, chloride, methyl sulfate, formate or acetate.

Teeth stand in the context of the present invention for natural or imitated teeth.

Teeth has the meaning of tooth and teeth.

In the context of the present invention fluorescent whitening agents encompass all fluorescent whitening agents known in the prior art. The definitions and preferences of fluorescent whitening agents given in the present invention are identical for the compositions comprising fluorescent whitening agents, and their uses.

The fluorescent whitening agents used according to the invention exhibit a marked antimicrobial effect, in particular against pathogenic Gram-positive and Gram-negative bacteria and also against skin flora bacteria. They are therefore suitable, in particular, for the disinfection, deodorization, and also the general and antimicrobial treatment of the skin and mucosae, and skin appendages (hair), very particularly for hand and wound disinfection. They are therefore suitable as antimicrobial active substances and preservatives in bodycare compositions, such as, for example, shampoos, tooth past, bath products, haircare compositions, liquid and solid soaps (based on synthetic surfactants and salts of saturated and/or unsaturated fatty acids), lotions and creams, deodorants, other aqueous or alcoholic solutions, e.g. cleansing solutions for the skin, moist cleansing wipes, oils or powders.

The invention therefore further provides a bodycare composition comprising at least one compound of the formula (1) and cosmetically acceptable carriers or auxiliaries.

The bodycare composition according to the invention comprises 0.01 to 15% by weight, preferably 0.1 to 10% by weight, based on the total weight of the composition, of fluorescent whitening agents and cosmetically acceptable auxiliaries.

Tooth paste according to the invention comprises 0.01 to 15% by weight, preferably 0.1 to 10% by weight, based on the total weight of the composition, of fluorescent whitening agents and cosmetically acceptable auxiliaries.

Depending on the form in which the bodycare composition is present, as well as the fluorescent whitening agents, it also has further constituents, such as, for example, sequestering agents, dyes, perfume oils, thickening or setting agents (consistency regulators), emollients, UV-absorbers, skin protectants, antioxidants, additives which improve the mechanical properties, such as dicarboxylic acids and/or Al, Zn, Ca, Mg salts of C₁₄-C₂₂ fatty acids and optionally preservatives.

The bodycare composition according to the invention can be formulated as a water-in-oil emulsion or oil-in-water emulsion, as an alcoholic or alcohol-containing formulation, as a vesicular dispersion of an ionic or nonionic amphiphilic lipid, as a gel, solid stick or as an aerosol formulation.

As a water-in-oil or oil-in-water emulsion, the cosmetically acceptable auxiliary preferably comprises 5 to 50% of an oil phase, 5 to 20% of an emulsifier and 30 to 90% of water. The oil phase can comprise any oil suitable for cosmetic formulations, such as, for example, one or more hydrocarbon oils, a wax, a natural oil, a silicone oil, a fatty acid ester or a fatty alcohol. Preferred mono- or polyols are ethanol, isopropanol, propylene glycol, hexylene glycol, glycerol and sorbitol.

Cosmetic formulations according to the invention are used in various fields. In particular, the following compositions, for example, are considered:
skincare compositions, such as, for example, skin washing and cleansing compositions in the form of bar or liquid soaps, syndets or washing pastes,
bath preparations, such as, for example, liquid bath preparations (foam baths, milks, shower preparations) or solid bath preparations, such as, for example, bath tablets and bath salts;
skincare compositions, such as, for example, skin emulsions, multiple emulsions or skin oils;
decorative bodycare compositions, such as, for example, make-up for the face in the form of day or powder creams, face powder (loose and pressed), blusher or cream make-up, eyecare compositions, such as, for example, eyeshadow preparations, mascara, eyeliner, eye creams or eye-fix creams; lipcare compositions, such as, for example, lipstick, lip gloss, lip liner pencil, nailcare compositions, such as nail varnish, nail varnish remover, nail hardeners, or cuticle removers;
intimate care compositions, such as, for example, intimate washing lotions or intimate sprays;
footcare compositions, such as, for example, foot baths, foot powders, foot creams or foot balsams, especially deodorants and antiperspirants or compositions for removing hard skin;
sunscreens, such as sun milks, lotions, creams, oils, sunblocks or tropicals, pretanning preparations or aftersun preparations;
skin-tanning preparations, such as, for example, self-tanning creams;
depigmentation compositions, such as, for example, skin bleaching preparations or skin lightening compositions;
insect-repelling compositions (“repellants”), such as, for example, insect oils, lotions, sprays or sticks;
deodorants, such as deodorant sprays, pump sprays, deodorant gels, sticks or roll-ons;
antiperspirants, such as, for example, antiperspirant sticks, creams or roll-ons;
compositions for the cleansing and care of blemished skin such as, for example, syndets (solid or liquid), peeling or scrub preparations or peeling masks;
hair-removal compositions in chemical form (depilation), such as, for example, hair-removal powders, liquid depilatories, cream or paste depilatories, depilatories in gel form or aerosol foams;
shaving compositions, such as, for example, shaving soap, foaming shaving creams, nonfoaming shaving creams, foams and gels, preshave preparations for dry shaving, aftershaves or aftershave lotions;
fragrances, such as, for example, toilet waters (eau de Cologne, eau de toilette, eau de parfum, parfum de toilette, perfume), perfume oils or perfume creams;
compositions for dental care, denture care and oral care, such as, for example, tooth creams, gel tooth creams, tooth powders, mouthwash concentrates, antiplaque mouthwashes, prothesis cleaners or prothesis adhesives;
cosmetic compositions for hair treatment, such as, for example, hair cleansers in the form of shampoos, hair conditioners, haircare compositions, such as, for example, pretreatment compositions, hair tonic, styling creams, styling gels, pomades, hair rinses, treatment packs, intensive hair treatments, compositions for shaping the hair, such as, for example, waving agents for producing permanent wave (hot-wave, mild-wave, coldwave), hair-smoothing preparations, liquid hair-setting compositions, hair foams, hair sprays, blonding agents, such as, for example, hydrogen peroxide solutions, lightening shampoos, blonding creams, blonding powders, blonding pastes or oils, temporary, semipermanent or permanent hair colorants, preparations with self-oxidizing dyes, or natural hair colorants, such as henna or chamomile.

An antimicrobial, whitening soap has, for example, the following composition:

| | | |
|---|---|---|
| 0.01 to 5% | by weight | of the compound of the formula (1) |
| 0.3 to 1% | by weight | of titanium dioxide, |
| 1 to 10% | by weight | of stearic stearic acid |

| | |
|---|---|
| ad 100% | of soap base, such as, for example, the sodium |
| | salts of tallow fatty acid and coconut fatty acid |
| | or glycerol. |

A shampoo has, for example, the following composition:

| 0.01 to 5% | by weight | of the compound of the formula (1), |
|---|---|---|
| 12.0% | by weight | of sodium laureth-2 sulphate, |
| 4.0% | by weight | of cocamidopropylbetaine, |
| 3.0% | by weight | of NaCl and |

| | |
|---|---|
| ad 100% | of water. |

A deodorant has, for example, the following composition:

| 0.01 to 5% | by weight | of the compound of the formula (1), |
|---|---|---|
| 60% | by weight | of ethanol, |
| 0.3% | by weight | of perfume oil, and |

| | |
|---|---|
| ad 100% | of water. |

The invention further provides an oral composition comprising 0.01 to 15% by weight, based on the total weight of the composition, of the compound of the formula (1) and orally acceptable auxiliaries.

Example of an oral composition:

| | | |
|---|---|---|
| 10% | by weight | of sorbitol, |
| 10% | by weight | of glycerol, |
| 15% | by weight | of ethanol, |
| 15% | by weight | of propylene glycol, |
| 0.5% | by weight | of sodium lauryl sulphate, |
| 0.25% | by weight | of sodium methyl cocyltaurate, |
| 0.25% | by weight | of polyoxypropylene/polyoxyethylene block |
| | | copolymer, |
| 0.10% | by weight | of peppermint flavouring, |
| 0.1 to 0.5% | by weight | of a compound of the formula (I), and |
| 48.6% | by weight | of water. |

The oral composition according to the invention can be, for example, in the form of a gel, a paste, a cream or an aqueous preparation (mouthwash).

In addition, the oral composition according to the invention can comprise compounds which release fluoride ions, which are effective against the formation of caries, e.g. inorganic fluoride salts, such as, for example, sodium fluoride, potassium fluoride, ammonium fluoride or calcium fluoride, or organic fluoride salts, such as, for example, amine fluorides, which are known under the trade name Olafluor.

In addition, the benzyl alcohol derivatives of the formula (1) used according to the invention are suitable for the treatment, in particular preservation, of textile fibre materials. The fibre materials are undyed and dyed or printed and are made of, for example, silk, wool, polyamide or polyurethanes, and in particular cellulosic fibre materials of all types. Such fibre materials are, for example, natural cellulose fibres, such as cotton, linen, jute and hemp, and also regenerated cellulose. Preferred suitable textile fibre materials are made of cotton.

The fluorescent whitening agents are also suitable for the treatment, in particular for the antimicrobial finishing or preservation, of plastics, such as, for example, polyethylene, polypropylene, polyurethane, polyester, polyamide, polycarbonate, latex etc. Fields of use for these are, for example, floor coverings, plastic coatings, plastic container and packaging materials; kitchen and bathroom utensils (e.g. brushes, shower curtains; sponges, bathroom mats), latex, filter materials (air and water filters), plastic articles used in the medical sector, such as, for example, bandaging materials, syringes, catheters etc., so-called medical devices, gloves and mattresses.

Paper too, such as, for example, hygiene papers, can be antimicrobially finished with the benzyl alcohols according to the invention.

In addition, nonwovens, such as, for example, nappies, sanitary towels, panty liners, wipes for the hygiene and household sector, can be antimicrobially finished according to the invention.

In addition, the fluorescent whitening agents are used in washing and cleaning formulations, such as, for example, in liquid and powder detergents or fabric softeners.

The fluorescent whitening agents can be used, in particular, also in household and all-purpose cleaners for the cleaning, whitening and disinfection of hard surfaces. A cleaner has, for example, the following composition:

| | |
|---|---|
| 0.01 to 5% | of the compound of the formula (1) |
| 3.0% | of octyl alcohol 4EO |
| 1.3% | of fatty alcohol C₈-C₁₀ polyglucoside |
| 3.0% | of isopropanol |
| ad 100% | of water. |

As well as the preservation and whitening of cosmetics and household products, the whitening, preservation and antimicrobial finishing of technical products and also use as biocide in technical processes is also possible, such as, for example, in the treatment of paper, in particular in paper-treatment liquors, printing thickeners made of starch or cellulose modifications, surface coatings and paints.

The fluorescent whitening agents are also suitable for the antimicrobial treatment of wood and also for the antimicrobial treatment, preservation and finishing of leather.

In addition, the compounds according to the invention are suitable for protecting cosmetic products and household products against microbial decay.

The fluorescent whitening agents which can be used according to the invention are known compounds.

### EXAMPLE 1

### Treatment of Hydroxyapatite with Fluorescent Whitening Agent

Solution A: 0.5% by weight of compound of formula (26) is solved in 100 g anhydrous ethanol.

Solution B: 0.1 g % by weight of compound of formula (26) is solved in 100 g anhydrous ethanol.

Solution C: 0.01% by weight of compound of formula (26) is solved in 100 g anhydrous ethanol.

Process a):

The hydroxyapatite substrate (seize ⅜″×0.06″, supplier Clarkson Chemical Co., USA) is given in the solution A (as given above), for 5 minutes. Then the hydroxyapatite substrate is taken out of the solution, shaken for removing the remaining solution and then dried at room temperature for 2 hours. For analytical purpose the hydroxyapatite substrate is then washed with distilled waster and dried in the air at room temperature for 18 hours.

Process b):

The process a) is repeated, with the proviso the solution A is exchanged by solution B.

Process c):

The process a) is repeated, with the proviso the solution A is exchanged by solution C.

### EXAMPLE 2

### Qualitative Prove of Fluorescence on the Treated Hydroxyapatite Substrate of Example 1

The fluorescence of the hydroxyapatite substrate prepared according to example 1 is determined with UV-light (254 nm). There is a fluorescence visible on the substrate. The intensity of the fluorescence is proportional to the used concentration of the solution A, B or C. The fluorescent whitening agent has affinity to the hydroxyapatite substrate since fluorescence is visible though the substrate is washed.

### EXAMPLE 3

### Whiteness (According Ganz) of the Treated Hydroxyapatite Substrate of Example 1

For evaluating the whiteness, the whiteness of the treated hydroxyapatite substrate is determined and than compared with that of the untreated hydroxy apatith substrate. There is a significant whitening visible, which is proportional to the used concentration of the solution A, B or C.

Used solution A, B or C

| | none | Solution C | Solution B | Solution A |
|---|---|---|---|---|
| Degree of | 82.3 | 87.1 | 91.2 | 96.6 |
| whiteness | | | | |

### EXAMPLE 4

### Determination of Minimum Inhibitory Concentration (MIC Values) in the Agar Incorporation Test

Test Principle:

1% stock solutions of the substances are prepared in an appropriate solvent and diluted in serial dilutions 1:2 (to yield end concentrations in the agar of 500-1.9 ppm). 0.3 ml of each dilution step is mixed with 15 ml of nutrient medium while the latter is still liquid. After the nutrient medium has solidified, 10 μl of each test strain in 0.85% NaCl solution are spotted onto the agar medium.

The plates are incubated at 37° C. for 24 hours and then the highest dilution (lowest concentration) of the test substance at which growth is no longer observable is determined.

| | |
|---|---|
| Nutrient medium: | Mueller Hinton agar (Difco) |
| | *Sabouraud 4% Glucose agar (Merck) |
| Diluent: | sterile 0.85% (w/w) NaCl solution |
| Incubation: | 24 hours at 37° C. |
| | *2-3 days at 29° C. |

Test Organisms:
Staphylococcus aureus ATCC 6583
Staphylococcus epidermidis ATCC 12228
Corynebacterium xerosis ATCC 373
Propionibacterium acnes ATCC
Escherichia coli ATCC 10536
Salmonella choleraesuis ATCC
Klebsiella pneumoniae ATCC
*Candida albicans ATCC 10231

*Aspergillus niger ATCC 6275

TABLE 1
MIC-values of bis-styryl-benzenes [ppm]

| Bis-styryl- | Staphylo- | | | Staphylo- |
|---|---|---|---|---|
| benzene | coccus | Corynebacterium | Corynebacterium | coccus |
| of formula | aureus | xerosis | minutissimum | epidermis |
| 23 | 7.8 | 7.8 | 3.9 | 15.6 |
| 24 | 7.5 | 3.8 | 3.8 | 7.5 |
| 25 | 62.5 | 15.6 | 15.6 | 62.5 |
| 26 | 6.3 | 3.1 | 6.3 | 6.3 |
| 27 | 12.5 | 3.1 | 3.1 | 12.5 |
| 28 | 6.3 | 6.3 | 3.1 | 6.3 |
| 29 | 3.1 | 3.1 | 6.3 | 3.1 |
| 30 | 12.5 | 3.1 | 3.1 | 12.5 |
| 31 | 62.5 | 125 | 15.6 | 125 |
| 32 | 0 | 100 | 200 | 50 |
| 33 | 3.9 | 31.3 | 7.8 | 3.9 |
| 34 | 3.1 | 3.1 | 3.1 | 3.1 |

| Bis- | | | | |
|---|---|---|---|---|
| styryl- | | | | |
| benzene | | | | |
| of | Propionibacterium | Escherichia | Salmonella | Klebsiella |
| formula | acnes | coli | choleraesuis | pneumoniae |
| 23 | 3.9 | 125 | 125 | 31.3 |
| 24 | 3.8 | 0 | 0 | 120 |
| 25 | 62.5 | 125 | 125 | 0 |
| 26 | 12.5 | 200 | 200 | 25 |
| 27 | 6.3 | 0 | 200 | 25 |
| 28 | 3.1 | 0 | 0 | 0 |
| 29 | 3.1 | 200 | 200 | 25 |
| 30 | 6.3 | 0 | 200 | 25 |
| 31 | 7.8 | 0 | 0 | 0 |
| 32 | 100 | 0 | 0 | 0 |
| 33 | 3.9 | 0 | 0 | 0 |
| 34 | 1.6 | 50 | 0 | 0 |

### EXAMPLE 5

### Determination of the Minimum Inhibition Concentration (MIC Value) Against Different Oral Microorganisms Via Broth Dilution

Test Principle:

1500 ppm stock solutions of the substances are prepared in ethanol and pipetted into the growth medium to yield concentrations between 0.94 and 15 ppm. Bacteria are taken from blood agar plates with cotton swabs and adjusted in the appropriate growth medium to yield an optical density corresponding to McFarland 0.5. This suspension is directly used in the case of F. nucleatum and P. nigrescens. For the other strains, the suspension is diluted 1:20. 0.1 ml of these bacterial suspensions are added to 2 ml of the substance solutions. After the incubation time, the tubes are assessed for growth (turbidity).

| | |
|---|---|
| Nutrient | Thioglycolate bouillon containing hemine and menadione |
| medium: | Columbia bouillon with hemine and menadione for |
| | P. gingivalis and P. nigrescens |
| Diluent: | the corresponding amount of stock solution was directly |
| | added to the growth medium |
| Incubation: | 7-10 days at 37° C. anaerobically |
| | *24 h aerobically with 10% CO₂ for Streptococci and |
| | A. actinomycetemcomintans |

Test Organisms:
*Actinobacillus actinomycetemcomitans ATCC 43718
*Stretococcus gordonii ATCC 10558
*Streptococcus mutans ATCC 33402
Actinomyces viscosus ATCC 43146
Fusobacterium nucleatum subsp. polymorphum ATCC 10953
Porphyromonas gingivalis ATCC 33277

Prevotella nigrescens ATCC 33563

TABLE 2
MIC-values of bis-styryl-benzenes [ppm]

| Bis-styryl- | | Strepto- | Strepto- | |
|---|---|---|---|---|
| benzene of | Actinomyces | coccus | coccus | Actinobacillus |
| formula | viscosus | gordonii | mutans | actinomycetemcomitans |
| 23 | 3.75 | 7.5 | 7.5 | 0 |
| 24 | 3.75 | 7.5 | 7.5 | 15 |
| 25 | 15 | 0 | 0 | 0 |
| 26 | 3.8 | 7.5 | 3.8 | 15 |
| 27 | 7.5 | 15 | 15 | 0 |
| 28 | 3.8 | 7.5 | 7.5 | 0 |
| 29 | 3.8 | 7.5 | 3.8 | 7.5 |
| 30 | 3.8 | 15 | 7.5 | 15 |
| 31 | 3.8 | 11.25 | 7.5 | 0 |
| 32 | 0 | 0 | 0 | 0 |
| 33 | 3.8 | 3.75 | 1.88 | 0 |
| 34 | 1.9 | 15 | 1.9 | 0 |

| Bis-styryl- | Fusobacterium | | |
|---|---|---|---|
| benzene of | nucleatum subsp. | Porphyromnas | Prevotella |
| formula | polymorphum | gingivalis | nigrescens |
| 23 | 7.5 | 3.75 | 7.5 |
| 24 | 15 | 1.9 | 1.9 |
| 25 | 0 | 0 | 15 |
| 26 | 3.8 | 3.8 | 1.9 |
| 27 | 7.5 | 7.5 | 3.8 |
| 28 | 15 | 1.9 | 3.8 |
| 29 | 3.8 | 1.9 | 1.9 |
| 30 | 3.8 | 3.8 | 3.8 |
| 31 | 15 | 1.9 | 3.75 |
| 32 | 0 | 0 | 0 |
| 33 | 0 | 15 | 3.75 |
| 34 | 0 | 7.5 | 3.8 |

### EXAMPLE 6

### Determination of Microbicidal Activity

Test Principle:

1 g stock solution with an appropriate concentration of test products are mixed with 8 g water and then inoculated with 1 ml of the selected test organisms. After a given contact period, aliquots are taken, inactivated and diluted. The number of surviving bacteria per ml incubation assay is determined by plate count. Proper inactivation by the inactivating medium used was checked each time.

Diluent: tryptone water for microorganisms

(0.1% tryptone (Oxoid), 0.85% NaCl, deion. water)
deion. water for test substances
inactivating medium for detection of surviving microorganisms
   Growth medium: casein soybean peptone agar
   Inactivating Medium: tryptic soy broth special
(10% w/w Tween 80, 3% w/w Lecithine, 0.1% w/w L-Histidine,
0.055% w/w Sodium thiosulfate)
   Test organisms: Staphylococcus aureus ATCC 6538
Escherichia coli ATCC 10536
Actinomyces viscosus ATCC 43146
Corynebacterium xerosis ATCC 373
   Test concentration: 120 ppm and 1200 ppm
   Contact times: 5 and 30 minutes at 22° C.

Incubation time: 24 h at 37° C.

TABLE 3
Microbicidal activity of styryl benzenes [log reduction]:

| Bis-styryl- | Staphylococcus aureus | | | | Escherichia coli | | | |
|---|---|---|---|---|---|---|---|---|
| benzene | 120 ppm/ | 120 ppm/ | 1200 ppm/ | 1200 ppm/ | 120 ppm/ | 120 ppm/ | 1200 ppm/ | 1200 ppm/ |
| of formula | 5 min | 30 min | 5 min | 30 min | 5 min | 30 min | 5 min | 30 min |
| 23 | 2 | 4 | | | 3.3 | 5 | | |
| 25 | 1 | 1 | 2.9 | 2.8 | 5 | 5 | 5 | 5 |
| 26 | 0 | 4 | | | 4.5 | 5 | | |
| 29 | 3 | 5 | | | 5 | 5 | | |

Microbicidal activity of bis styryl benzenes [log reductions]

| Bis-styryl- | Actinomyces viscosus | | | | Corynebacterium xerosis | | | |
|---|---|---|---|---|---|---|---|---|
| benzene | 120 ppm/ | 120 ppm/ | 1200 ppm/ | 1200 ppm/ | 120 ppm/ | 120 ppm/ | 1200 ppm/ | 1200 ppm/ |
| of formula | 5 min | 30 min | 5 min | 30 min | 5 min | 30 min | 5 min | 30 min |
| 23 | 2 | 4 | | | 5 | 5 | | |
| 25 | 2.9 | 2.1 | 4 | 4 | 5 | 5 | 5 | 5 |
| 26 | 0 | 3.7 | | | 2.9 | 5 | | |
| 29 | 2.5 | 5 | | | 5 | 5 | | |

### EXAMPLE 7

### Substantivity on Hydroxyapatite and Determination of Growth Inhibition

Test Principle:

Hydroxyapatite discs are incubated in artificial saliva (German Dental Magazine DZZ 5/2002) for 4 hrs under stirring, rinsed in NaCl, dried over night, and then incubated in ethanolic solutions of the test substances. Then all treated discs are put in 12 well Nucleon surface titre plates (one disc per well), and Caso Broth inoculated with the test strain. The titre plates are incubated at 37° C., samples are taken after 6 and 24 hrs and the colony count is determined by plate count.

| | |
|---|---|
| Diluent: | 0.85% (w/w) NaCl |
| | ethanol for test substances |
| Medium: | casein soybean peptone agar |
| Test organism: | Actinomyces viscosus ATCC 43146 |
| Test concentration: | 500 ppm |
| Contact times: | 6 and 24 hours at 37° C. |
| Incubation time: | 24-48 h at 37° C. |

The illustrations show the growth inhibition of Actinomyces viscosus by the bis-styryl benzenes of formula (24) and (33) after adsorption of the substances on hydroxyapatite discs, that were pretreated with artificial saliva in comparison to an untreated control.

1. A method for the antimicrobial treatment of surfaces comprising applying thereto said surfaces at least one fluorescent whitening agent.

2. A method according to claim 1 for whitening and antimicrobial treatment of surfaces.

3. A method according to claim 2 for for whitening and antimicrobial treatment of teeth.

4. A method according to claim 1, wherein the fluorescent whitening agent is bis-triazinyl-diaminostilbene, 2-(stilbene-4-yl)naphthatriazole, 2-(4-phenylstilbene-4-yl)benzoxazole, bis(azol-2-yl)stilbene, 1,4-bis(styryl)benzene, 4,4′-bis(styryl)biphenyl, 1,3-diphenyl-2-pyrazoline, bis(benzoxazol-2-yl), bis(benzimidazol-2-yl), 2-(benzofuran-2-yl)-benzimidazole, coumarine, carbostyrile, naphthalimide, quaternized pyridotriazole, pyrene derivatives or acylamino 3,7-diamino-dibenzothiophene-2,8-disulfonic acid 5,5-dioxide.

5. A method according to claim 4, wherein the fluorescent whitening agent is a compound of formulae (1) to (20): bis-triazinyl-diaminostilbene of formula (1) 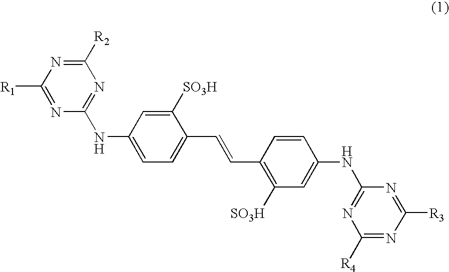
wherein
R₁, R₂, R₃ and R₄ are independently from each other NR₅R₆, OR₇ or a heterocyclic ring
wherein
R₅ and R₆ are independently from each other hydrogen; substituted or unsubstituted C₆-C₁₀aryl, C₁-C₁₀alkyl, N,N′-diC₁-C₆alkylaminoC₁-C₁₀alkyl or a heterocyclic ring, and
R₇ is substituted or unsubstituted C₆-C₁₀aryl, C₁-C₁₀alkyl;
or
2-(stilbene-4-yl)-naphthatriazole of formula (2) 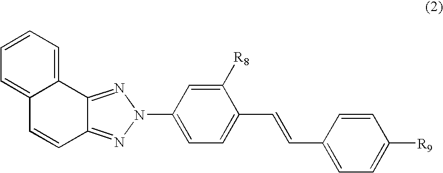
wherein
R₈ and R₉ are independently from each other hydrogen, SO₃H, CN, halogen;
or
2-(4-phenylstilbene-4-yl)benzoxazole of formula (3) 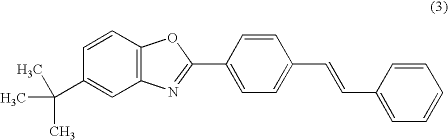
or
bis(azol-2-yl)stilbene of formula (4) 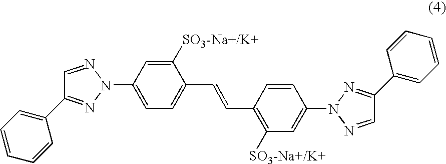
or
1,4-bis(styryl)benzene of formula (5) 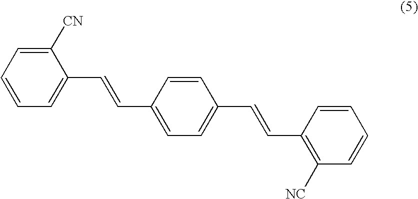
or
4,4-bis(styryl)biphenyl of formula (6) 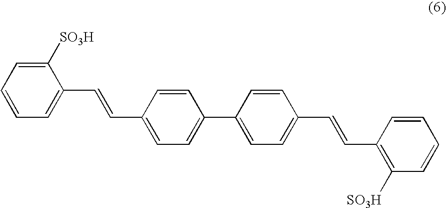
or
1,3-diphenyl-2-pyrazoline of formula (7) 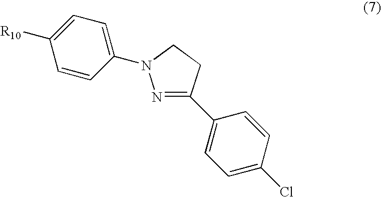
R₁₀ is SO₃H, SO₂NR₁₁R₁₂,
wherein
R₁₁ and R₁₂ are each independently from each other hydrogen, (C₁-C₆)alkyl-N⁺(C₁-C₆)alkyl, (C₁-C₆)alkyl-SO₃H;
or
bis-benzoxazole of formula (8) 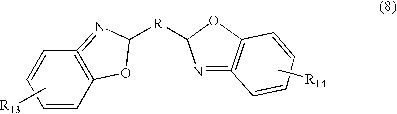
wherein
R₁₃ and R₁₄ are independently from each other hydrogen; substituted or unsubstituted C₆-C₁₀aryl, C₁-C₁₀alkyl, 1,2-diphenylvinyl, COO—C₁-C₁₀alkyl or SO₂—C₁-C₁₀alkyl, and
R is —C═C—, 1,2-dipenylvinylen, 1,4-naphthalen or 2,5-thiophenylene;
or
bis(benzimidazol-2-yl) of formula (9) 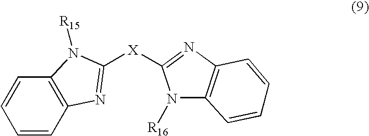
wherein
R₁₅ and R₁₆ are independently from each other hydrogen substituted or unsubstituted C₁-C₁₆alkyl or phenyl; and 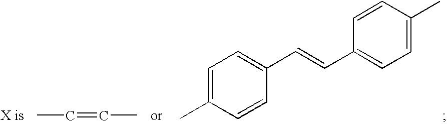
or
2-(benzofuran-2-yl)-benzimidazole of formula (10) 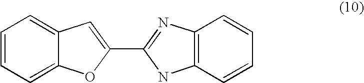
or
coumarine, including 3-phenyl-7-aminocoumarin, 3-phenyl-7-(azol-2-yl)coumarines, 3,7-bis(azolyl)coumarines, and compounds of formulae (11), (12), (13) or (14) 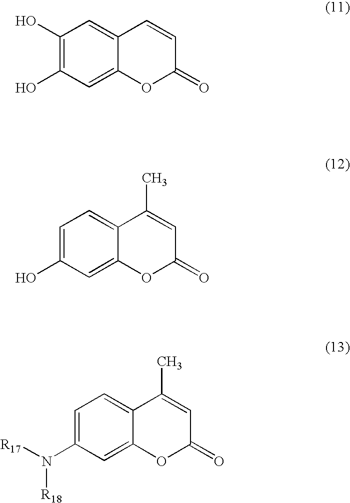
wherein
R₁₇ and R₁₈ are independently from each other hydrogen or substituted or unsubstituted C₁-C₆alkyl 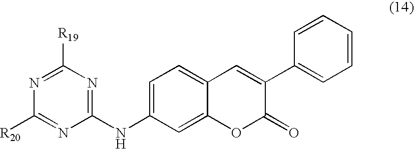
wherein
R₁₉ and R₂₀ are independently from each other NR₂₁R₂₂, OR₂₃ or a heterocyclic ring
wherein
R₂₁ and R₂₂ are independently from each other hydrogen; substituted or unsubstituted C₆-C₁₀aryl, C₁-C₁₀alkyl and
R₂₃ is substituted or unsubstituted C₆-C₁₀aryl, C₁-C₁₀alkyl;
or
carbostyrile of formula (15) 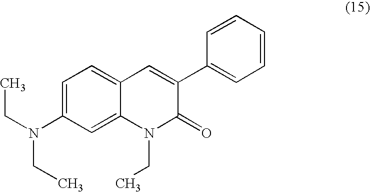
or
naphthalimide of formula (16) 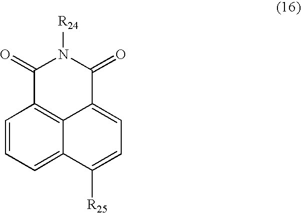
wherein
R₂₄ is hydrogen or substituted or unsubstituted C₁-C₁₆alkyl or phenyl, and
wherein
R₂₅ is hydrogen or substituted or unsubstituted NR₂₆R₂₇, OR₂₈ or a heterocyclic ring
wherein
R₂₆, R₂₇ and R₂₈ have the same definition as R₂₁, R₂₂ and R₂₃ as given above; or quaternized pyridotriazole;
or
pyrene of formula (17) 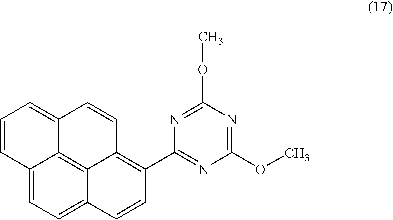
or
acylamino 3,7-diamino-dibenzothiophene-2,8-disulfonic acid 5,5-dioxide of formula (18) 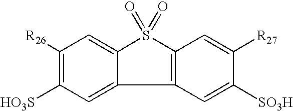
wherein
R₂₆ and R₂₇ are independently from each other substituted or unsubstituted CO-alkoxybenzoyl, CO—(C₁-C₆)alkyl, CO-phenyl,
or
bisstyrylbenzol of formula (19) 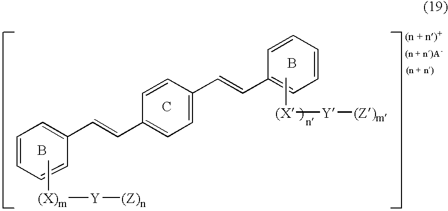
or
bisstyrylbiphenyl of formula (20) 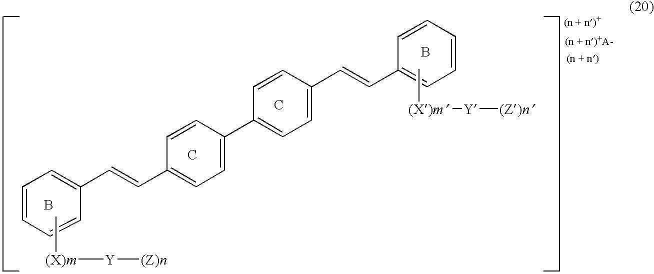
wherein
X and X′ independently of one another are —COO— or —CON(R₃₁), a direct bond, oxygen, sulfur, —O—C₁-C₃alkylene-CON(R₃₁)—, —SO₂N(R₃₁)—, —O—C₁-C₃-alkylene-COO— or —OCO—,
Y and Y′ independently of one another are a direct bond, C₁-C₂₀alkylene, a direct bond,
Z is pyridine, 2-pyridine-N-methyl, 4-pyridine-N-methyl or N(R₂₈R₂₉(R₃₀)_{q}), and
Z′ is pyridine, 2-pyridine-N-methyl, 4-pyridine-N-methyl or N(R_{28′}R_{29′}(R₃₀)_{q′}),
wherein
R₂₈ and R_{28′} independently of one another are unsubstituted or substituted C₁-C₈-alkyl or
C₃-C₄alkenyl, or R₂₈ together with R₂₉, or R_{28′} together with R_{29′}, is a heterocyclic ring, R₂₉ and R_{29′} independently of one another are unsubstituted or substituted C₁-C₈alkyl or C₃-C₄alkenyl, or R₂₉ together with R₂₈ or R_{29′} together with R_{28′}, is a heterocyclic ring, or R₂₈ and R₂₉, or R_{28′} and R_{29′}, together with R₃₀ are a pyridine or picoline ring, R₃₀ is hydrogen, unsubstituted or substituted C₁-C₄alkyl or C₃-C₄alkenyl, or together with R₂₈ and R₂₉ or with R_{28′} and R_{29′} is a pyridine or picoline ring,
R₃₀ is hydrogen or unsubstituted or substituted C₁-C₆-alkyl,
A⁻ is a colourless anion, and
n and n′ independently of one another are the number 0 or 1, and
m and m′ independently of one another are the number 0 or 1, and
p and p′ independently of one another are the number 0, 1, 2 or 3, and
q and q′ independently of one another are the number 0 or 1, and
the benzene nuclei B and C can also be substituted by non-chromophoric substituents.

6. A method according to claim 4, wherein the fluorescent whitening agent is bisstyrylbenzol of formula (21) 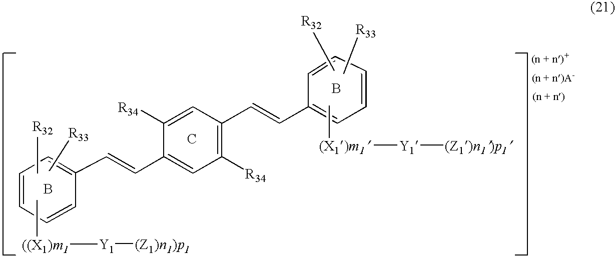
or
bisstyrylbiphenyl of formula (22) 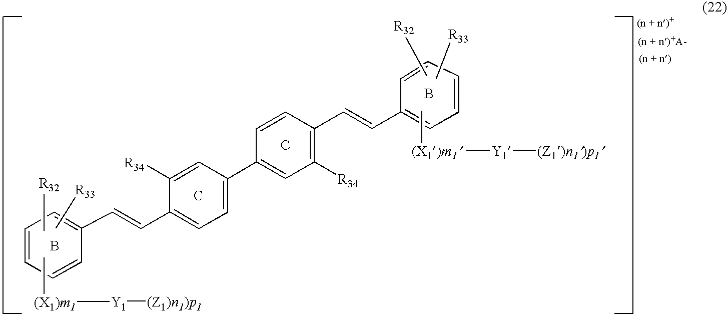
wherein
X₁ and X₁′ are —COO— or —CONH—, a direct bond, oxygen, sulfur, —OC₁-C₃alkylene-CONH—, —SO₂NH—, —O—C₁-C₃alkylene-COO— or —OCO—,
Y₁ and Y_{1′} independently of one another are a direct bond, C₁-C₄alkylene or hydroxypropylene,
Z₁ and Z₁′ independently of one another are pyridine, 2-pyridine-N-methyl, 4-pyridine-N-methyl or N(R₃₅R₃₆(R₃₇)_{q″}),
wherein
R₃₅ and R₃₆ independently of one another are C₁-C₄alkyl or together are a pyrrolidine, piperidine, hexamethyleneimine or morpholine ring, or together with R₃₇ are a pyridine or picoline ring,
R₃₀ is hydrogen, C₁-C₄alkyl, C₃-C₄alkenyl, C₁-C₃alkoxycarbonylmethyl, benzyl, C₂-C₄-hydroxyalkyl or C₂-C₄cyanoalkyl, or together with R₃₅ and R₃₆ is a pyridine or picoline ring,
R₃₂ is hydrogen, chlorine, C₁-C₄alkyl, C₃-C₄alkenyl, C₁-C₃alkoxy, or (X₁)ₘ—Y₁—N(R₃₅R₃₆(R₃₇)_{q″}), or together with R₃₃ is a trimethylene or tetramethylene group,
R₃₃ is hydrogen, chlorine, C₁-C₄alkyl or C₁-C₃alkoxy, or together with R₃₂ is a trimethylene or tetramethylene group,
R₃₄ is hydrogen, chlorine or methyl,
n₁ and n_{1′} independently of one another are the number 0 or 1,
m₁ and m₁′ independently of one another are the number 0 or 1, and
p₁ and p₁′ independently of one another are the number 0, 1, 2 or 3, and
q″ is the number 0 or 1, and
A⁻ is a colourless anion.

7. A method according to claim 4, wherein the fluorescent whitening agent is of formulae (23) to (34)

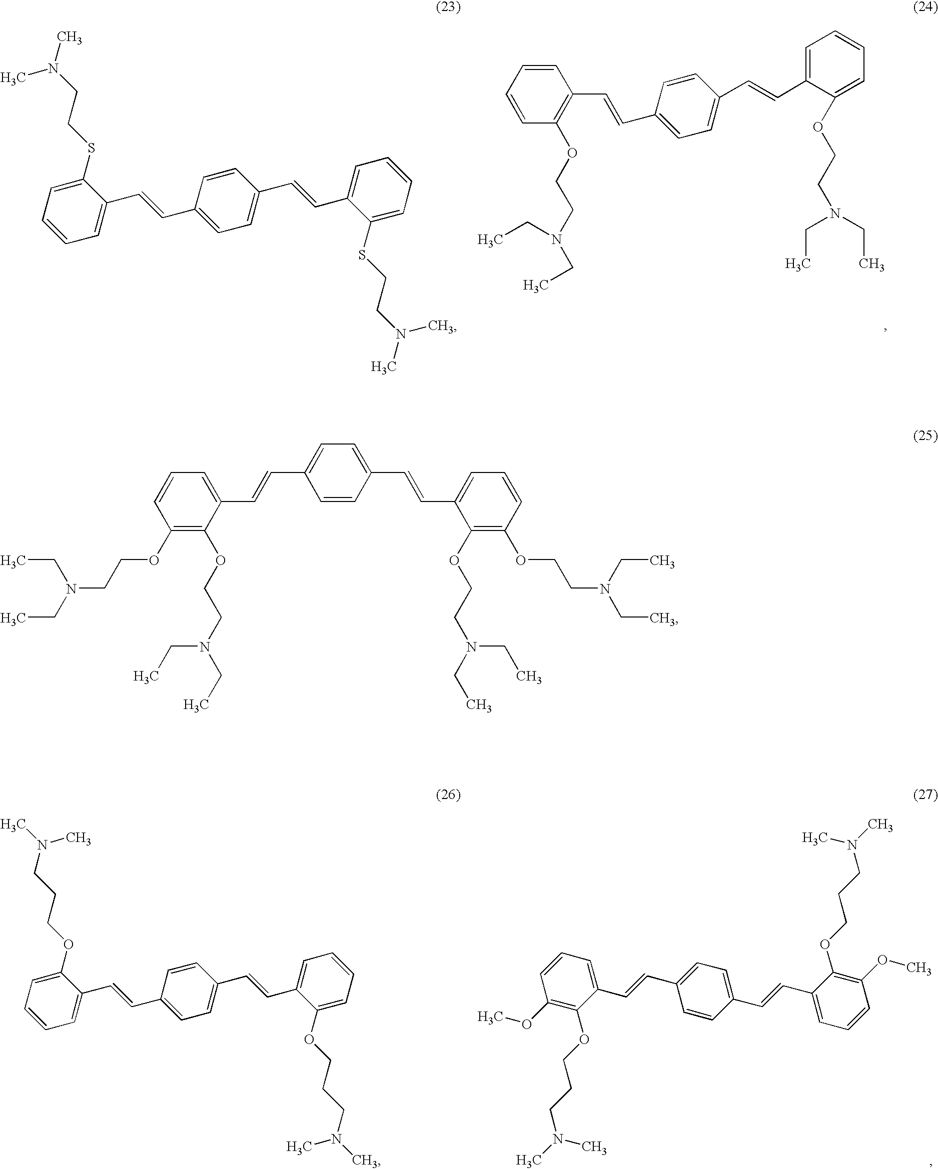

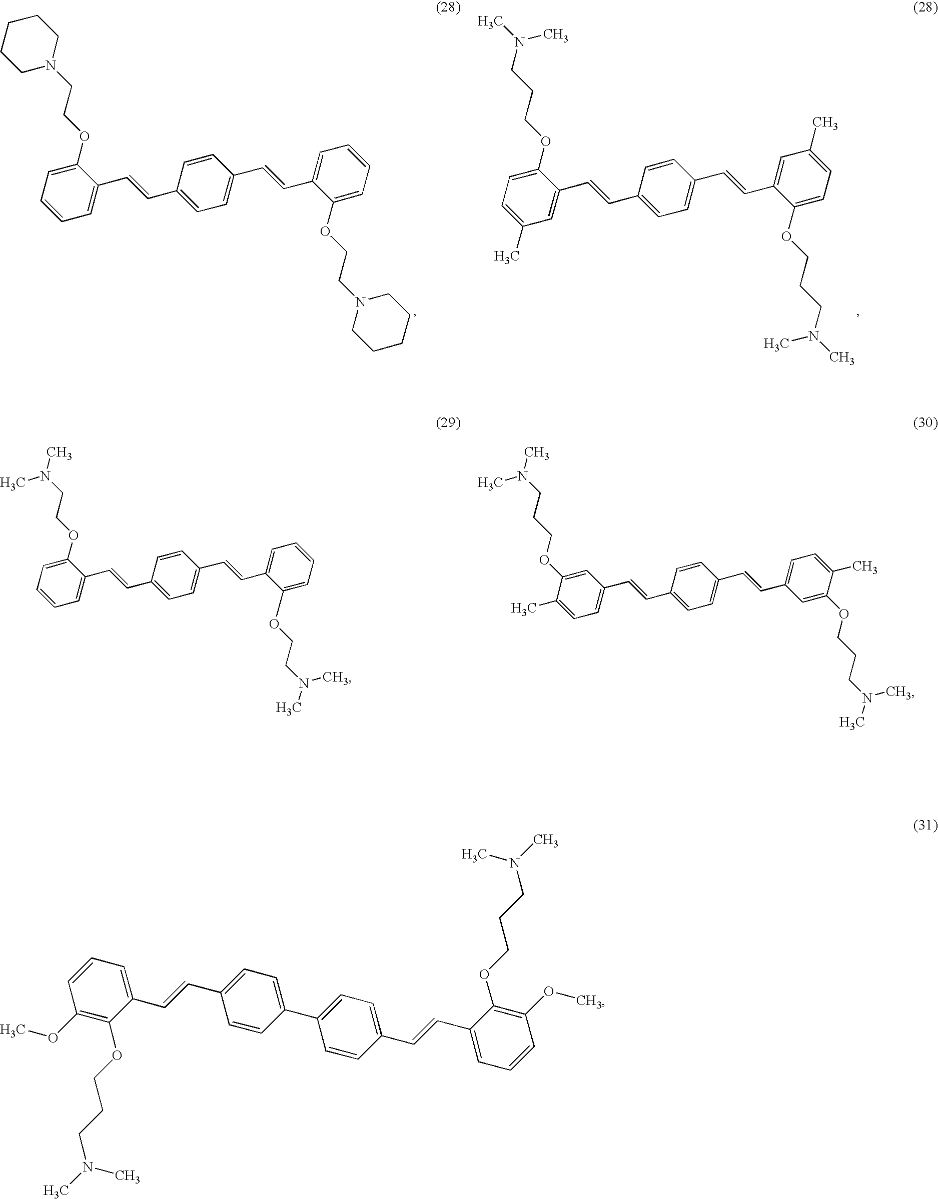

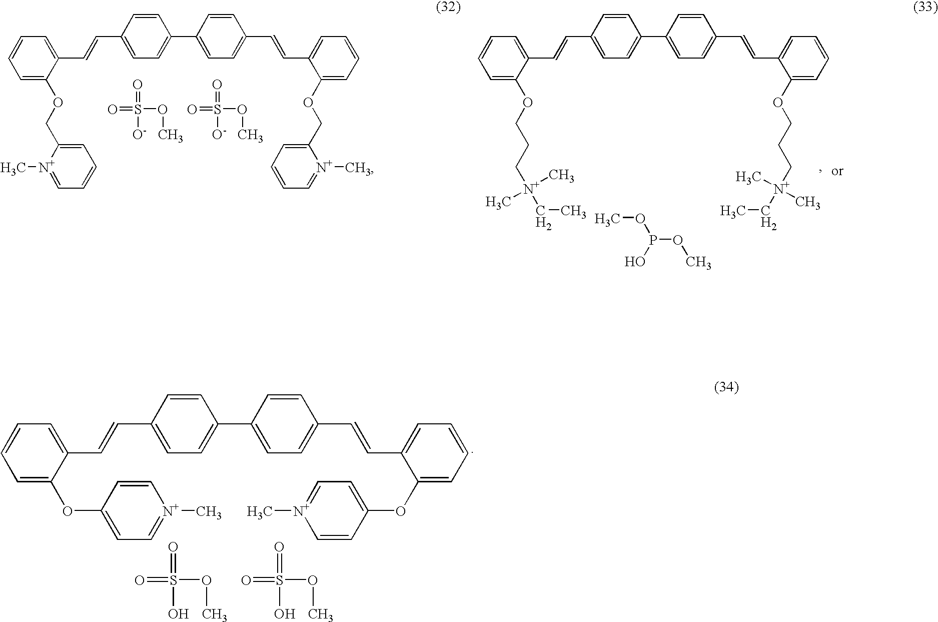

8. A method according to claim 1 for the whitening and antimicrobial treatment of dental care products, denture care and oral care products.

9. A method according to claim 8, wherein at least one fluorescent whitening agent is comprised in a composition, said composition is tooth cream, gel tooth cream, tooth powder, mouthwash concentrate, antiplaque mouthwashes, prothesis cleaners or prothesis adhesives.

10. A method according to claim 1 for the antimicrobial treatment, deodorization, disinfections and/or whitening of the skin, mucosae and hair.

11. A method according to claim 10 for disinfections and deodorization of the skin, mucosae and hair.

12. A method according to claim 1 for the antimicrobial treatment and whitening of textile fibre materials.

13. A method according to claim 1 for preservation and/or whitening of surfaces.

14. A method according to claim 1 for the antimicrobial treatment and whitening in washing and cleaning formulations.

15. A method according to claim 1 for the whitening and antimicrobial finishing and preservation of compositions selected from the group consisting of plastics, paper, nonwovens, wood and leather.

16. A method according to claim 1 for the whitening and antimicrobial finishing and preservation of technical products selected from the group consisting of printing thickeners made of starch or cellulose modifications, surface coatings and paints.

17. A method according to claim 1 wherein the fluorescent whitening agent is a biocide in technical processes.

18. A bodycare composition comprising
0.01 to 15% by weight, based on the total weight of the composition, of at least one fluorescent whitening agent and cosmetically acceptable auxiliaries.

19. An oral composition according to claim 18 comprising 0.01 to 15% by weight, based on the total weight of the composition, of at least one fluorescent whitening agent and orally acceptable auxiliaries.

20. A tooth paste according to claim 18 comprising 0.01 to 15% by weight, based on the total weight of the composition, of at least one fluorescent whitening agent and orally acceptable auxiliaries.
